(19) United States
(12) Patent Application Publication
(10) Pub. No.: US 2017/0045721 A1
(43) Pub. Date: Feb. 16, 2017

(54) MICROSCOPE AUTOFOCUS FOR RETINAL SURGERY

(71) Applicant: Novartis AG, Basel (CH)

(72) Inventor: Steven T. Charles, Memphis, TN (US)

(21) Appl. No.: 14/824,235

(22) Filed: Aug. 12, 2015

(51) Int. Cl. G02B 21/00; A61B 3/10; A61B 3/13

(52) U.S. Cl. CPC G02B 21/0012; A61B 3/13; A61B 3/102

(57) ABSTRACT

The present invention provides an autofocus surgical microscope system that comprises a surgical microscope, a time-of-flight module, a focus controller, and a focus mechanism. The focus controller is coupled to the time-of-flight module. The focus mechanism is coupled to the focus controller and the surgical microscope. The time-of-flight module determines a distance to an eye structure, and the focus controller controls the focus mechanism based on the determined distance to focus the surgical microscope on the eye structure

### BACKGROUND OF THE INVENTION

The present invention relates to using optical time-of-flight technology to focus a microscope during retinal surgery.

Ophthalmic surgery is typically performed under an operating microscope. The microscope is used to visualize various structures in the eye. For example, during retinal surgery, the microscope is used to visualize structures in the posterior segment such as the retina, various membranes, and the vitreous. During cataract surgery, the microscope is used to visualize structures in the anterior segment such as the lens and capsular bag. In both posterior segment surgery and anterior segment surgery, it is important to control the microscope to properly visual these small eye structures. Typically, the surgeon focuses the microscope manually on these eye structures.

One of the most difficult posterior segment procedures is peeling the internal limiting membrane (ILM) from the retina. The ILM must be peeled away from the retinal surface in virtually all macular surgery cases, for example, full & partial thickness macular hole, vitreomacular traction syndrome, epimacular membrane, and vitreomacular schisis. The ILM is three microns thick, colorless, transparent, and featureless. Successful ILM peeling without damaging the underlying retina requires critical focus of the operating microscope and high magnification. During retinal surgery, patients often repetitively move their head up and down with respiratory motion. Such head movement is especially common with overweight patients and those with sleep apnea, COPD, or congestive heart failure. Auto-focus using high spatial frequencies cannot be used to focus on the ILM because it is transparent and featureless. Ultrasound cannot be used to focus motorized optical elements in the operating microscope because of insufficient resolution and other surgical-limiting factors. It would be desirable to automatically focus the microscope on the ILM during membrane peeling.

In cataract surgery, the natural lens is removed from the eye and an artificial lens is implanted in its place. In order to gain access to the natural lens, an opening is made in the capsular bag in a procedure known as a capsulorhexis. The capsular bag, like the ILM, is a very thin, transparent membrane. The same issues arise in focusing the microscope on the capsular bag. It would be desirable to automatically focus the microscope on the capsular bag during a capsulorhexis.

### SUMMARY OF THE INVENTION

In one embodiment consistent with the principles of the present invention, the present invention is a method of focusing a surgical microscope comprising: sensing a position of an eye structure; determining a distance to the eye structure; and focusing the surgical microscope on the eye structure based on the determined distance. The method may also comprise sending the determined distance to a focus controller and controlling a focus mechanism based on the determined distance. Sensing the position of the eye structure may also comprise using a time-of-flight module to sense the position of the eye structure. The time-of-flight module may be an OCT system, a LIDAR system, or an optical range finder. In some cases, the time-of-flight module is an OCT system that uses an optical signal to determine the distance to the eye structure. The time-of-flight module may send an optical signal through an optical path of the microscope. The method may also comprise receiving in the focus controller the determined distance and sending a control signal to the focus mechanism. The method may further comprise compensating for movement of the eye structure by continuously focusing the surgical microscope on the eye structure.

In another embodiment consistent with the principles of the present invention, the present invention is an autofocus surgical microscope system comprising: a surgical microscope; a time-of-flight module; a focus controller coupled to the time-of-flight module; a focus mechanism coupled to the focus controller and the surgical microscope; wherein the time-of-flight module determines a distance to an eye structure, and the focus controller controls the focus mechanism based on the determined distance to focus the surgical microscope on the eye structure. The time-of-flight module may be an OCT system, a LIDAR system, or an optical range finder. In some cases, the time-of-flight module is an OCT system that uses an optical signal to determine the distance to the eye structure. The time-of-flight module may send an optical signal through an optical path of the surgical microscope. In some cases, the focus controller is programmed to receive an input signal from the time-of-flight module, analyze the received input signal, and produce an output signal that causes the focus mechanism to focus the surgical microscope. The focus mechanism may comprise a motor. The time-of-flight module is configured to sense a position of the eye structure. The focus controller is configured to compensate for movement of the eye structure by continuously controlling the focus mechanism to focus the surgical microscope on the eye structure.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a schematic diagram of a surgical console.

FIG. 2 is a cross section view of an eye and a vitrectomy probe.

FIG. 3 is a block diagram of a time-of-flight autofocus system for a surgical microscope.

FIG. 4 is a block diagram of a time-of-flight autofocus system for a surgical microscope.

FIG. 5 is a method of continuously focusing a microscope on an eye structure.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

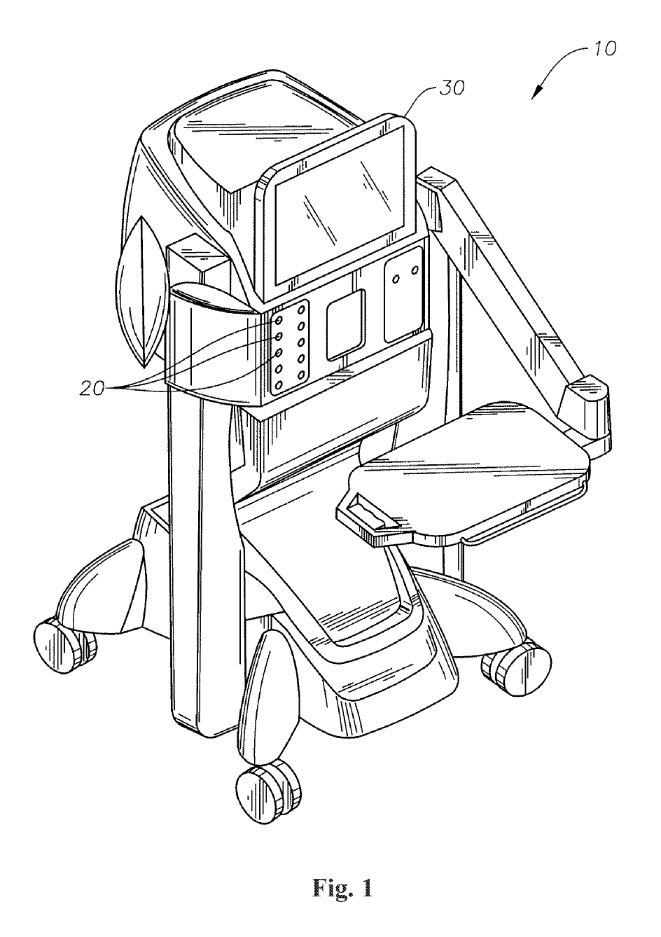

FIG. 1 shows an example surgical console 10. The surgical console may be a vitreoretinal surgical console, such as the Constellation® surgical console produced by Alcon Laboratories, Inc., 6201 South Freeway, Fort Worth, Tex. 76134 U.S.A. The console 10 may include one or more ports 20. One or more of the ports 20 may be utilized for providing infusion and/or irrigation fluids to the eye or for aspirating materials from the eye. The console 10 may also include a display 30 for interfacing with the console 10, such as to establish or change one or more operations of the console 10. In some instances, the display 30 may include a touch-sensitive screen for interacting with the console 10 by touching the screen of the display 30. A probe, such as a vitrectomy probe may be coupled to a port 20 for dissecting ocular tissues and aspirating the ocular tissues from the eye. A similar surgical console is used during cataract surgery.

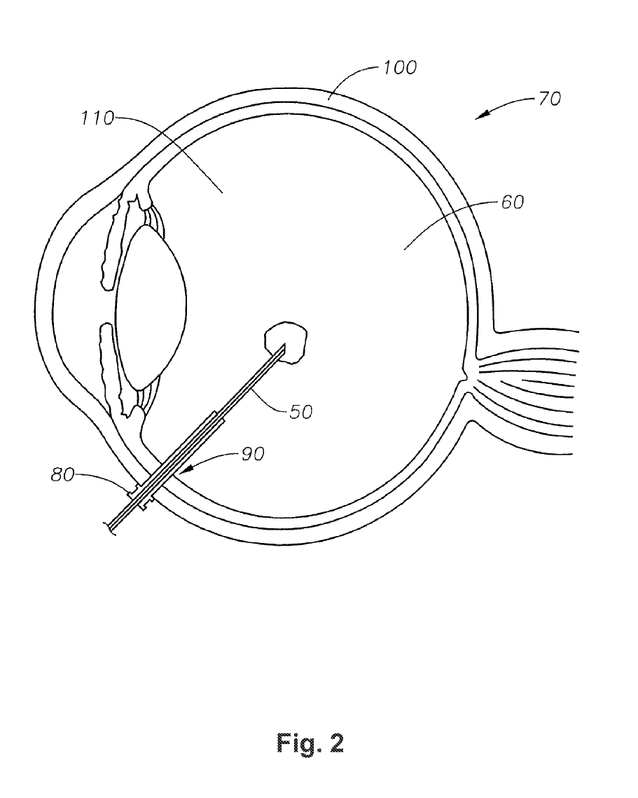

As illustrated in FIG. 2, during an ophthalmic surgical procedure, such as a retinal surgical procedure, a vitrectomy probe 50 may be inserted into the posterior segment 60 of the eye 70, such as through a cannula 80 disposed in an incision 90 through the sclera 100 of the eye 70, to remove and aspirate ocular tissues. For example, during a retinal surgical procedure, the vitrectomy probe 50 may be inserted into the posterior chamber 60 of the eye 70 to remove vitreous humor (also referred to as “vitreous”) 110, a jelly-like substance that occupies the volume defined by the posterior segment 60. The vitrectomy probe 50 may also be used to remove membranes covering the retina or other tissues. A blade and forceps may be inserted through cannula 80 to dissect and peel the ILM.

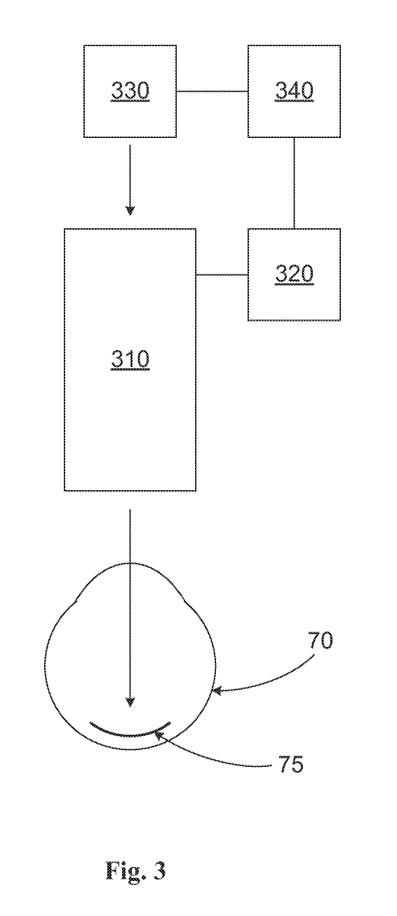

FIG. 3 is a block diagram of a time-of-flight autofocus system for a surgical microscope. During a retinal surgical procedure, a microscope 310 is used to visualize the structures in the posterior segment 60 of eye 70. For example, during surgery, a microscope 310 is focused on a particular eye structure 75, such as the ILM. In the system depicted in FIG. 3, a microscope 310 is focused on an eye structure 75 in eye 70. Time-of-flight module 330 is coupled to focus controller 340. Focus controller 340 is coupled to focus mechanism 320. Focus mechanism 320 is coupled to microscope 310. In this example, focus mechanism 320 is a motor, such as a stepper motor, that is coupled to the microscope 310 in such a way as to allow focus mechanism 320 to focus the microscope 310. When a motor is used as focus mechanism 320, the motor shaft may be coupled to the focus dial on microscope 310. When the motor is actuated, the motor shaft turns the focus dial on microscope 310. In another example, focus mechanism may comprise other types of actuators that can be used to focus the microscope 310 by causing the optics in microscope 310 to move. For example, focus mechanism 320 may be coupled directly to the optics of microscope 310 or the structures that support the optics of microscope 310. In this manner, focus mechanism 320 causes the optics of microscope 310 to move so as to focus microscope 310. Preferably, focus mechanism 320 is an actuator that acts quickly so as to keep microscope 310 focused on a moving object.

In the example of FIG. 3, focus controller 340 controls focus mechanism 340. Focus controller 340 receives a signal from time-of-flight module 330, and uses that signal to control focus mechanism 320. Focus controller 340 is typically a microcontroller, microprocessor, or other similar device. Focus controller 340 is programmed to receive a signal from time-of-flight module 330, analyze that signal, and produce an output signal that causes focus mechanism 320 to focus microscope 310.

In the example of FIG. 3, time-of-flight module 330 utilizes optical time-of-flight technology to sense the position of an eye structure 75 in eye 70. In one example, time-of-flight module 330 sends an optical signal through the microscope optics and into the eye 70 as depicted by the arrows in FIG. 3. The optical signal is reflected back off of the eye structure 75 in eye 70 to time-of-flight module 330. In this manner, time-of-flight module 330 is able to determine the distance to the eye structure 75. This distance is then sent to focus controller 340 to control focus mechanism 320. In this manner, time-of-flight module 330 measures the distance to an eye structure 75 in eye 70 and sends that distance measurement to focus controller 340. Focus controller 340 uses that distance measurement to control focus mechanism 320 such that microscope 310 remains focused on the eye structure 75 being sensed by time-of-flight module 330. When the eye structure 75 moves (for example, because of head movement or eye rotation), the system of FIG. 3 is able to keep the microscope focused on that eye structure 75. In different examples, time-of-flight module 330 may be on optical coherence tomography system (OCT system), a Light Detection and Ranging system (LIDAR system), or an optical range finder. In each case, time-of-flight module 330 uses an optical signal to determine the distance to an eye structure 75 being sensed.

In operation, the system of FIG. 3 keeps the microscope 310 focused on the eye structure 75 being sensed. Initially, the microscope 310 is focused on an eye structure 75 either manually or automatically. The time-of-flight module 330 continuously senses the position of the eye structure 75 using optical signals. The time-of-flight module 330 continuously determines the distance to the eye structure 75 based on the optical signals. In one example, the eye structure 75 is the ILM. The time-of-flight module 330 continuously senses the position of the ILM with a series of optical signals and continuously determines the distance to the ILM based on those optical signals. This distance measurement is sent to focus controller 340. Focus controller 340 controls focus mechanism 320 to keep the microscope 310 continuously focused on the eye structure 75 based on the distance measurement. The system continues to sense the position of the eye structure 75, determine a distance to the eye structure 75, and focus of the microscope 310 on the eye structure 75.

In another example, the eye structure 75 is the capsular bag. The time-of-flight module 330 continuously senses the position of the capsular bag with a series of optical signals and continuously determines the distance to the capsular bag based on those optical signals. This distance measurement is sent to focus controller 340. Focus controller 340 controls focus mechanism 320 to keep the microscope 310 continuously focused on the eye structure 75 based on the distance measurement. The system continues to sense the position of the eye structure 75, determine a distance to the eye structure 75, and focus of the microscope 310 on the eye structure 75.

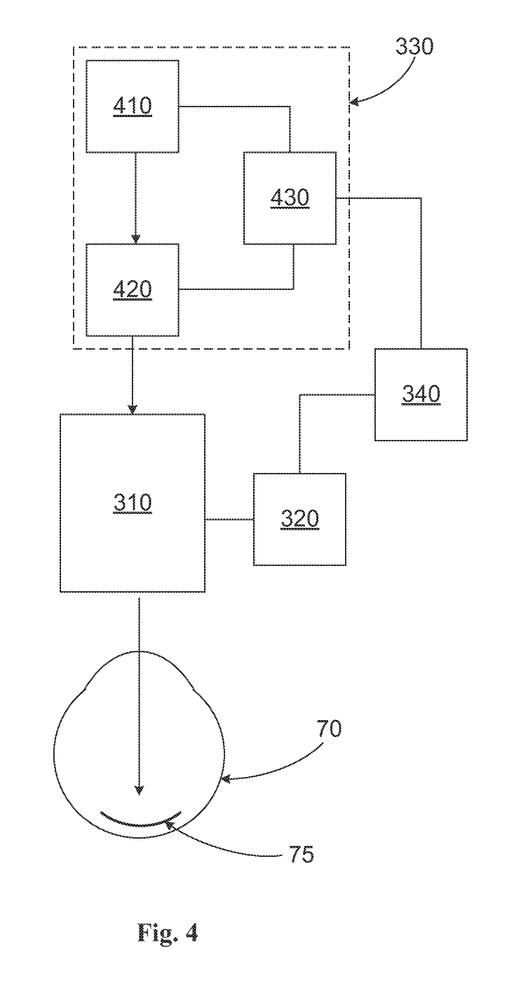

FIG. 4 is a block diagram of a time-of-flight autofocus system for a surgical microscope. In the example of FIG. 4, a microscope 310 is focused on an eye structure 75 in eye 70. Time-of-flight module 330 is coupled to focus controller 340. Focus controller 340 is coupled to focus mechanism 320. Focus mechanism 320 is coupled to microscope 310. In this example, time-of-flight module 330 is an OCT system and comprises an OCT light source 410, an OCT beam scanner 420, and an OCT controller 430. OCT light source 310 is typically a low power laser light source. OCT beam scanner 420 typically includes a scanning mirror, an actuator to move the scanning mirror, and associated optics. OCT controller 340 controls the operation of OCT light source 410 and OCT beam scanner 420 as well as providing other control functions. In one example, OCT controller 430 determines the distance to an eye structure 75 that is being sensed. OCT controller communicates this distance to focus controller 340 so that focus mechanism 320 can keep microscope 310 focused on the eye structure 75 being sensed.

In operation, the system of FIG. 4 keeps the microscope 310 focused on the eye structure 75 being sensed. Initially, the microscope 310 is focused on an eye structure 75 either manually or automatically. The OCT light source 410, OCT beam scanner 420, and OCT controller sense the position of the eye structure 75 by scanning light from OCT light source 410 on the eye structure 75. Based on this sensing, the distance to the eye structure 75 is determined. In one example, the eye structure 75 is the ILM. The OCT light source 410, OCT beam scanner 420, and OCT controller continuously sense the position of the ILM and continuously determine the distance to the ILM based on the scanned OCT light beam. This distance measurement is sent to focus controller 340. Focus controller 340 controls focus mechanism 320 to keep the microscope 310 continuously focused on the eye structure 75 based on the distance measurement. The system continues to sense the position of the eye structure 75, determine a distance to the eye structure 75, and focus of the microscope 310 on the eye structure 75.

In another example, the eye structure 75 is the capsular bag. The OCT light source 410, OCT beam scanner 420, and OCT controller continuously sense the position of the capsular bag and continuously determine the distance to the capsular bag based on the scanned OCT light beam. This distance measurement is sent to focus controller 340. Focus controller 340 controls focus mechanism 320 to keep the microscope 310 continuously focused on the eye structure 75 based on the distance measurement. The system continues to sense the position of the eye structure 75, determine a distance to the eye structure 75, and focus of the microscope 310 on the eye structure 75.

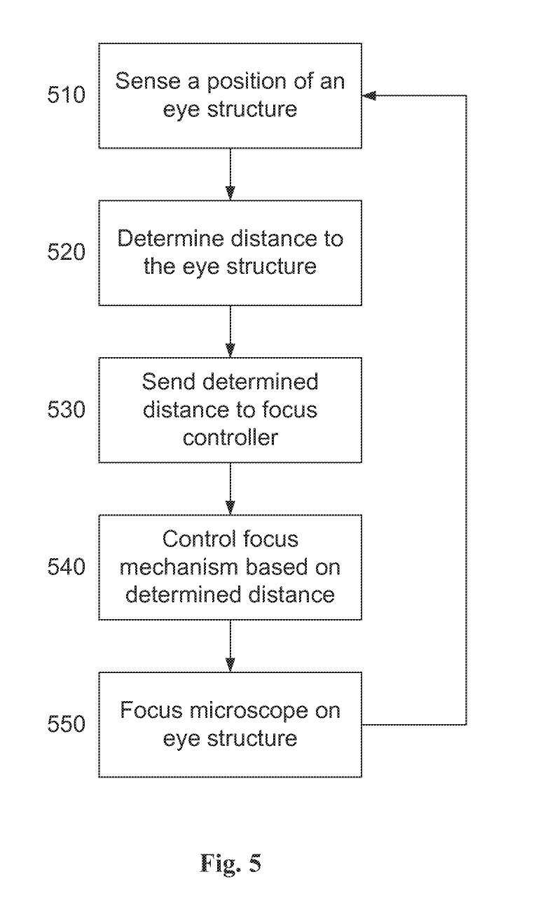

FIG. 5 is an example of a method of continuously focusing a microscope 310 on an eye structure 75. In 510, the position of the eye structure 75 is sensed, for example, by a time-of-flight imaging method. In 520, a distance to the eye structure 75 is determined. In 530, the determined distance is sent to a focus controller 340. In 540, the focus controller 340 controls a focus mechanism 320 based on the determined distance. In 550, the focus mechanism 320 causes the microscope 310 to focus on the eye structure 75. The same series of functions is then repeated to continuously focus the microscope 310 on the eye structure 75. In this manner, the microscope 310 can be continuously focused on the eye structure 75 even if the eye structure 75 moves.

From the above, it may be appreciated that the present invention provides an improved system for focusing a microscope on an eye structure during retinal surgery. The present invention provides a device and associated method for using a time-of-flight imaging system to determine a distance to an eye structure, providing the distance as a control input to a focus controller, and controlling a focus mechanism to continuously keep a microscope focused on the eye structure. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of focusing a surgical microscope, the method comprising:
sensing a position of an eye structure;
determining a distance to the eye structure; and
focusing the surgical microscope on the eye structure based on the determined distance.

2. The method of claim 1 further comprising:
sending the determined distance to a focus controller; and
controlling a focus mechanism based on the determined distance.

3. The method of claim 1 wherein sensing the position of the eye structure further comprises using a time-of-flight module to sense the position of the eye structure.

4. The method of claim 3 wherein the time-of-flight module is selected from the group consisting of: an OCT system, a LIDAR system, and an optical range finder.

5. The method of claim 3 wherein the time-of-flight module is an OCT system that uses an optical signal to determine the distance to the eye structure.

6. The method of claim 3 wherein the time-of-flight module sends an optical signal through an optical path of the microscope.

7. The method of claim 2 further comprising:
receiving in the focus controller the determined distance; and
sending a control signal to the focus mechanism.

8. The method of claim 1 further comprising:
compensating for movement of the eye structure by continuously focusing the surgical microscope on the eye structure.

9. An autofocus surgical microscope system comprising:
a surgical microscope;
a time-of-flight module;
a focus controller coupled to the time-of-flight module;
a focus mechanism coupled to the focus controller and the surgical microscope;
wherein the time-of-flight module determines a distance to an eye structure, and the focus controller controls the focus mechanism based on the determined distance to focus the surgical microscope on the eye structure.

10. The system of claim 9 wherein the time-of-flight module is selected from the group consisting of: an OCT system, a LIDAR system, and an optical range finder.

11. The system of claim 9 wherein the time-of-flight module is an OCT system that uses an optical signal to determine the distance to the eye structure.

12. The system of claim 9 wherein the time-of-flight module sends an optical signal through an optical path of the surgical microscope.

13. The system of claim 9 wherein the focus controller is programmed to receive an input signal from the time-of-flight module, analyze the received input signal, and produce an output signal that causes the focus mechanism to focus the surgical microscope.

14. The system of claim 9 wherein the focus mechanism comprises a motor.

15. The system of claim 9 wherein the time-of-flight module is configured to sense a position of the eye structure.

16. The system of claim 9 wherein the focus controller is configured to compensate for movement of the eye structure by continuously controlling the focus mechanism to focus the surgical microscope on the eye structure.
